(19) United States
(12) Patent Application Publication
(10) Pub. No.: US 2022/0395625 A1
(43) Pub. Date: Dec. 15, 2022

(54) SELF-CLEANING ELECTRIC NASAL ASPIRATOR

(71) Applicant: Chunling Ye, Ganzhou City (CN)

(72) Inventor: Chunling Ye, Ganzhou City (CN)

(21) Appl. No.: 17/586,171

(22) Filed: Jan. 27, 2022

(30) Foreign Application Priority Data
Jun. 11, 2021 (CN) 202121309527.4

(51) Int. Cl. A61M 1/00; B08B 9/035

(52) U.S. Cl. CPC A61M 1/64; B08B 9/035; A61M 2205/8206; A61M 2209/10

(57) ABSTRACT

Provided is a self-cleaning electric nasal aspirator. The self-cleaning electric nasal aspirator includes a suction assembly. The suction assembly includes a suction nozzle and a storage compartment. The suction nozzle is in communication with the storage compartment. The storage compartment is configured to temporarily store nasal mucus sucked out through the suction nozzle. The suction assembly further includes a suction compartment and a storage compartment cover. The storage compartment cover is disposed at an upper end of the storage compartment. A liquid storage space is formed by being enclosed by the storage compartment cover and the storage compartment. The suction compartment is sleeved outside the storage compartment and the storage compartment cover. The self-cleaning electric nasal aspirator can prevent nasal mucus from overflowing to the surface of the self-cleaning electric nasal aspirator and has a self-cleaning function.

### CROSS-REFERENCE TO RELATED APPLICATION(S)

This application claims priority to Chinese Patent Application No. 202121309527.4 filed Jun. 11, 2021, the disclosure of which is incorporated herein by reference in its entirety.

### TECHNICAL FIELD

The present disclosure relates to the technical field of mother and child care and, in particular, to a self-cleaning electric nasal aspirator.

### BACKGROUND

Children are prone to catch a cold accompanied by a runny nose due to a weak constitution. At the stage when children have not learned to blow their noses, parents often need to help clean up their noses. Otherwise, dry nasal mucus forms nasal scabs blocking the nasal cavity, thereby affecting the breathing of the children.

As an auxiliary tool, the nasal aspirator can aspirate nasal mucus in the nasal cavity of children. The most commonly used nasal aspirator is a pump-type nasal aspirator. The pump-type nasal aspirator is divided into two types: electric and manual. At present, there are many electric nasal aspirators on the market.

However, at present, most of the electric nasal aspirators on the market do not have a self-cleaning function. When an electric nasal aspirator is full of nasal mucus, the nasal mucus overflows and adheres to the surface of the electric nasal aspirator, and even penetrates into electronic components, thereby being not conducive to cleaning and long-term use.

Therefore, a self-cleaning electric nasal aspirator is urgently needed to overcome the preceding technical problems.

### SUMMARY

Based on the preceding, an object of the present disclosure is to provide a self-cleaning electric nasal aspirator. The self-cleaning electric nasal aspirator can prevent nasal mucus from overflowing to the surface of the self-cleaning electric nasal aspirator and has a self-cleaning function.

Provided is a self-cleaning electric nasal aspirator, including a suction assembly.

The suction assembly includes a suction nozzle and a storage compartment. The suction nozzle is in communication with the storage compartment. The storage compartment is configured to temporarily store nasal mucus sucked out through the suction nozzle. The suction assembly further includes a suction compartment and a storage compartment cover. The storage compartment cover is disposed at an upper end of the storage compartment. A liquid storage space is formed by being enclosed by the storage compartment cover and the storage compartment, and the suction compartment is sleeved outside the storage compartment and the storage compartment cover.

### BRIEF DESCRIPTION OF DRAWINGS

To illustrate solutions in embodiments of the present disclosure more clearly, drawings used in description of the embodiments of the present disclosure are briefly described below. Apparently, the drawings described below illustrate only part of the embodiments of the present disclosure, and those of ordinary skill in the art may obtain other drawings based on the content and drawings described below on the premise that no creative work is done.

FIG. 1 is a view illustrating the structure of a self-cleaning electric nasal aspirator according to the present disclosure;

FIG. 2 is an exploded view of a self-cleaning electric nasal aspirator according to the present disclosure;

FIG. 3 is a view illustrating the structure of a principal machine according to the present disclosure;

FIG. 4 is a view illustrating the structure of a connector according to the present disclosure; and

FIG. 5 is a partial exploded view of a suction assembly according to the present disclosure.

### REFERENCE LIST

100 principal machine
11 housing
111 state indicator
12 aspirator pump
121 suction port
122 discharge port
13 buffer compartment
14 connector
141 first pipe
142 second pipe
15 battery compartment
16 electronic control module
161 control circuit board
162 control button
17 outlet
18 charging module
181 type-c socket
200 suction assembly
21 suction nozzle
22 storage compartment
221 eduction pipe
222 liquid guiding passage
23 suction compartment
231 suction pipe
24 storage compartment cover
25 liquid storage space
26 first gap
27 second gap

### DETAILED DESCRIPTION

To illustrate technical problems, technical solutions, and technical effects of the present disclosure more clearly, the solutions of embodiments of the present disclosure are further described below in detail in conjunction with drawings. Apparently, embodiments described herein are merely part, not all, of the embodiments of the present disclosure. Based on the embodiments of the present disclosure, all other embodiments obtained by those skilled in the art without creative work are within the scope of the present disclosure.

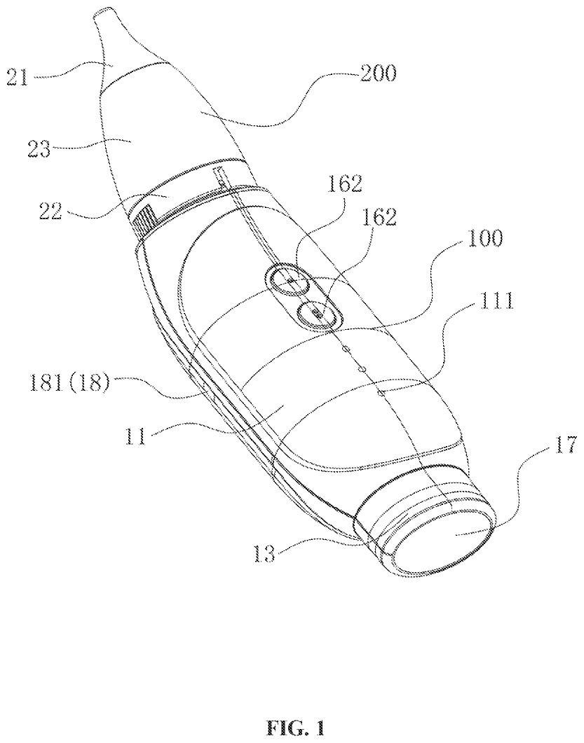

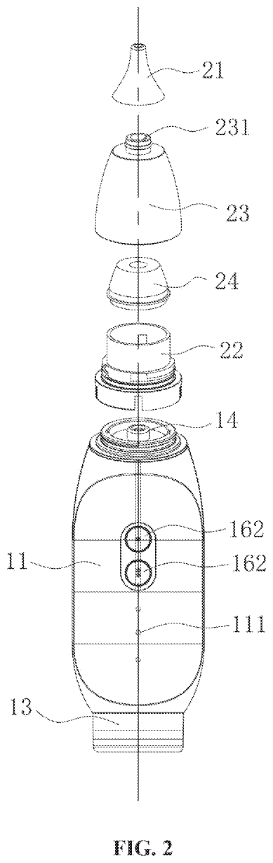

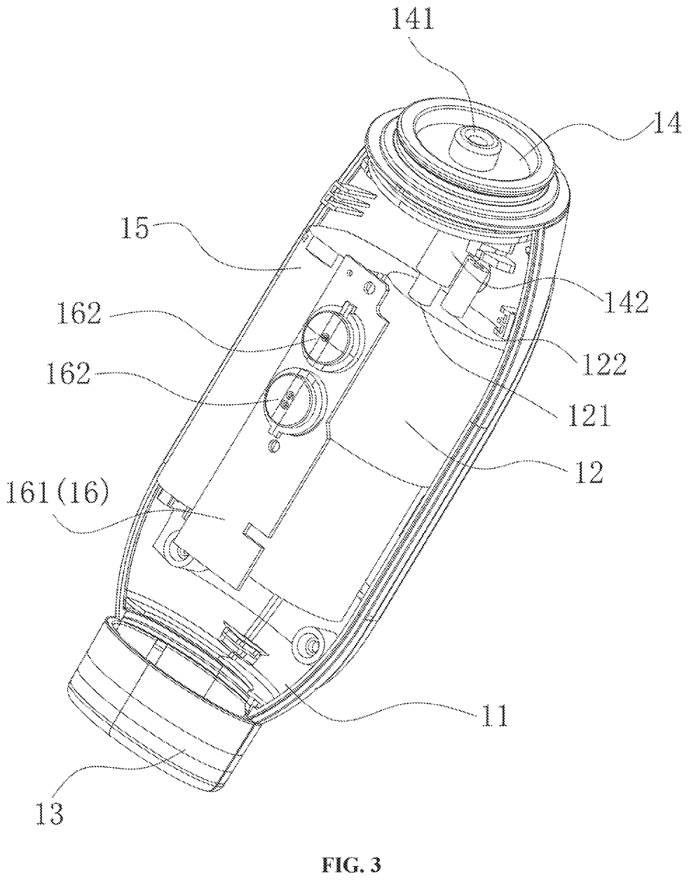

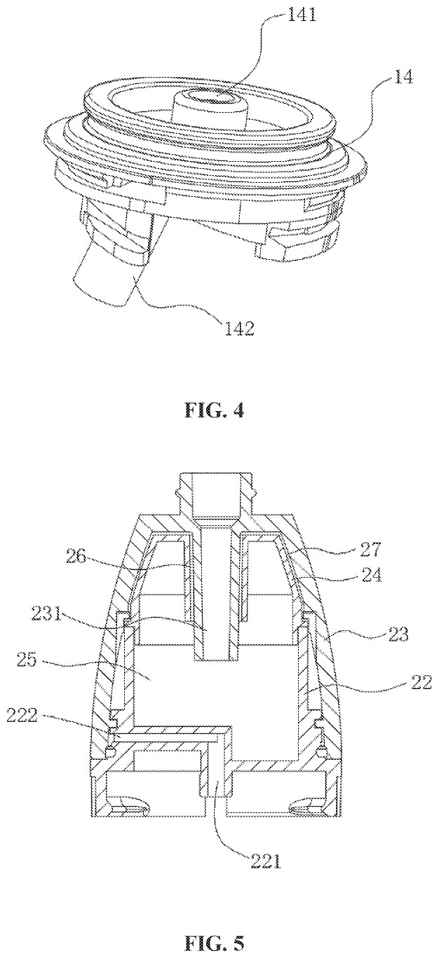

As shown in FIGS. 1 to 5, this embodiment provides a self-cleaning electric nasal aspirator for sucking nasal mucus in an infant nasal cavity. The self-cleaning electric nasal aspirator includes a principal machine 100 and a suction assembly 200. The principal machine 100 includes a housing 11, an aspirator pump 12 and a buffer compartment 13. The housing 11 is a hollow structure. The aspirator pump 12 is disposed in the cavity of the housing 11. The aspirator pump 12 is a water and gas dual-purpose pump. The buffer compartment 13 is disposed at the rear end of the housing 11. The buffer compartment 13 is connected to a discharge port 122 of the aspirator pump 12. The bottom of the principal machine 100 is provided with an outlet 17 through which the buffer compartment 13 is in communication with the outside of the principal machine 100. The suction assembly 200 is detachably connected to the front end of the principal machine 100. The suction assembly 200 includes a suction nozzle 21 and a storage compartment 22. The suction nozzle 21 is in communication with the storage compartment 22. The storage compartment 22 is in communication with the suction port 121 of the aspirator pump 12. The storage compartment 22 is configured to temporarily store nasal mucus sucked out through the suction nozzle 21. The aspirator pump 12 is configured to pump, into the buffer compartment 13, nasal mucus overflowing from the storage compartment 22.

The self-cleaning electric nasal aspirator provided in this embodiment can not only prevent the risk of infection caused by overflow of nasal mucus, but also has a self-cleaning function. When it is needed to suck nasal mucus, the self-cleaning electric nasal aspirator is started. Then the suction nozzle 21 is extended into a nasal cavity. At the initial stage, the self-cleaning electric nasal aspirator has no nasal mucus, the aspirator pump 12 is in an air suction state, and the nasal mucus in the nasal cavity is gradually sucked into the storage compartment 22. After a plurality of times of use, the storage compartment 22 is filled with nasal mucus, and the nasal mucus overflows from the storage compartment 22. At this time, the aspirator pump 12 continues sucking the nasal mucus from the storage compartment 22 and is in a liquid suction state. The aspirator pump 12 discharges the sucked nasal mucus from the discharge port 122 to the buffer compartment 13 through the pump body until the buffer compartment 13 is filled with nasal mucus. The nasal mucus is discharged from the outlet 17 at the bottom. Since the buffer compartment 13 and the outlet 17 are each disposed at the rear end of the self-cleaning electric nasal aspirator, the overflowing nasal mucus can not contaminate the outer surface of the self-cleaning electric nasal aspirator. The self-cleaning function is achieved simply in the following manner. When it is needed to clean the nasal mucus in the self-cleaning electric nasal aspirator, the suction nozzle 21 is put into clean water, the clean water is sucked through the aspirator pump 12, and then the nasal mucus is washed away by the flow of clean water in the self-cleaning electric nasal aspirator and discharged through an outlet 17 at the bottom of the principal machine 100. It is also possible to remove the whole of the suction assembly 200 from the principal machine 100 and clean the suction nozzle 21 and the storage compartment 22 separately, thereby improving the cleaning effect.

The water and gas dual-purpose pump used in the aspirator pump 12 in this embodiment is a micro vacuum air pump. A motor drives a mechanical device to make a diaphragm inside the pump reciprocate, thereby generating a vacuum by compressing and stretching air in the pump cavity having a fixed capacity. A pressure difference between the vacuum and the external atmospheric pressure is generated at a pump air extraction port. Under the action of the pressure difference, gas and liquid are sucked into the pump cavity from the suction port and then discharged from the discharge port. The working medium of this water and gas dual-purpose pump may be a gas or a liquid, which belongs to the related art and is repeated here.

Optionally, the suction port 121 of the aspirator pump 12 is provided with a filter mesh. The filter mesh can prevent large foreign objects from blocking the aspirator pump 12, and the liquid sucked into the pump body is isolated so that the liquid is prevented from entering the motor and damaging the motor.

Optionally, the housing 11 includes an upper housing and a lower housing, the upper housing is engaged to the lower housing are engaged, and the buffer compartment 13 is engaged at the rear end of the upper housing and the rear end of the lower housing.

Further, a drainage hose may be inserted at the outlet 17 to discharge nasal mucus during nasal aspiration or to discharge the cleaning liquid during self-cleaning.

Optionally, the principal machine 100 further includes a connector 14. The aspirator pump 12 is connected to the storage compartment 22 through the connector 14. The connector 14 is a hollow structure, and the connector 14 is provided with a first pipe 141 and a second pipe 142 in communication with the first pipe 141. The first pipe 141 is connected with, in a pluggable manner, an eduction pipe 221 at the bottom of the storage compartment 22, and the second pipe 142 is connected with the suction port 121 of the aspirator pump 12 in a pluggable manner.

Optionally, the suction assembly 200 further includes a suction compartment 23 and a storage compartment cover 24. The suction compartment 23 is provided with a suction pipe 231. The storage compartment cover 24 is disposed at the upper end of the storage compartment 22. A liquid storage space 25 is formed by being enclosed by the storage compartment cover 24 and the storage compartment 22. One end of the suction pipe 231 extends into the liquid storage space 25 through the storage compartment cover 24, the suction nozzle 21 is sleeved at the other end of the suction pipe 231, and the suction compartment 23 is sleeved outside the storage compartment 22 and outside the storage compartment cover 24.

Optionally, a first gap 26 is disposed between the suction pipe 231 and the storage compartment cover 24, and a second gap 27 is disposed between the outer wall of the storage compartment 22 and the inner wall of the suction compartment 23 and between the outer wall of the storage compartment cover 24 and the inner wall of the suction compartment 23. A liquid guiding passage 222 is disposed at the bottom of the storage compartment 22, and the liquid storage space 25 is in communication with the eduction pipe 221 through the first gap 26, the second gap 27 and the liquid guiding passage 222. When the nasal mucus in the liquid storage space 25 does not overflow into the first gap 26, the aspirator pump 12 is in the air suction state, and the air in the liquid guiding passage 222, the first gap 26, the second gap 27 and the liquid storage space 25 is extracted to the vacuum state, thereby forming a negative pressure in the liquid storage space 25 to suck the nasal mucus, so that the nasal mucus is sucked into the liquid storage space 25 from the nasal cavity through the suction nozzle 21 and the suction pipe 231 of the suction compartment 23. At this time, during the suction of the nasal mucus, the flow direction of the air and the liquid is opposite, the liquid is sucked into the bottom of the storage compartment cover 24 downwardly, and the air is sucked upwardly. Since the gravity of the liquid resists the negative pressure, the liquid does not follow the airflow to form a backflow, thereby preventing the nasal mucus from being easily sucked into the aspirator pump 12 along with the airflow when the air and liquid are in the same cavity, resulting in lower nasal mucus suction efficiency and easy damage to the pump body. When the nasal mucus in the liquid storage space 25 overflows to the first gap 26, according to the floating ball principle, the nasal mucus buoyancy pushes the storage compartment cover 24 and lifts the storage compartment cover 24 so that the top surface of the storage compartment cover 24 is in contact with the inner wall of the suction compartment 23. At this time, the airflow is blocked, and the negative pressure can no longer be formed in the liquid guiding passage 222, the first gap 26, the second gap 27 and the liquid storage space 25, and the suction is stopped automatically to prevent backflow and overflow, thereby effectively preventing the equipment from being damaged when the liquid exceeds the maximum capacity value and enters the pump body to damage the motor.

Under the continuous action of the aspirator pump 12, the nasal mucus is finally sucked into the aspirator pump 12 through the first gap 26, the second gap 27, the liquid guiding passage 222, the eduction pipe 221, the first pipe 141 and the second tube pipe 142, and then discharged to the buffer compartment 13.

Optionally, the suction compartment 23, the storage compartment 22, and the storage compartment cover 24 are each made of a transparent or translucent material so that users can observe the amount of nasal mucus in the storage compartment 22 from the outside to see in advance that the nasal mucus in the storage compartment 22 is about to overflow, and clean the storage compartment 22 in time, and thus the nasal mucus is prevented from being sucked into the aspirator pump 12 and the step of cleaning the aspirator pump 12 is avoided.

Optionally, the storage compartment 22 is made of a transparent polycarbonate (PC) material, and the connector 14 is made of a soft rubber material. In an example, the connector 14 is made of elastic and softer soft rubber so that the first pipe 141 and the eduction pipe 221 of the storage compartment 22 form a structure in which soft rubber covers hard rubber, and the second pipe 142 and the suction port 121 of the aspirator pump 12 form a structure in which soft rubber covers hard rubber. In this manner, the first pipe 141 is connected with, in a pluggable manner, the eduction pipe 221 of the storage compartment 22 more securely, between the second pipe 142 is connected with, in a pluggable manner, the suction port 121 of the aspirator pump 12 more securely, the sealing performance is better, and the risk of air leakage and liquid leakage is reduced.

Optionally, the suction nozzle 21 is made of soft silica gel, and the suction nozzle 21 is arranged in a shape with a small upper part and a large lower part so as to be easy to extend into the nasal cavity for operation.

Optionally, the principal machine 100 further includes a power supply module, and the power supply module includes a battery. The battery is configured to supply power to the aspirator pump 12. The back of the housing 11 is provided with a battery compartment 15, and the battery is accommodated in the battery compartment 15.

Further, the battery is a rechargeable battery, the principal machine 100 further includes a charging module 18, and the charging module 18 is configured to charge the battery. Moreover, the charging module 18 includes a type-c socket 181, the type-c socket 181 is disposed on the housing 11, and users can charge the battery through the type-c socket 181.

Optionally, the principal machine 100 further includes an electronic control module 16. The electronic control module 16 includes a control circuit board 161 and a control button 162. The control circuit board 161 is disposed in the cavity of the housing 11. The control circuit board 161 has a control circuit that can be loaded with a control program. The control circuit board 161 is electrically connected to the aspirator pump 12 and configured to control the action of the aspirator pump 12. The control button 162 is disposed on the control circuit board 161. The control button 162 penetrates through the front side of the housing 11. In an example, two control buttons 162 are provided. One of the two control buttons 162 is configured to control the switch of the self-cleaning electric nasal aspirator, and another of the two control buttons 162 is configured to control the gear of the self-cleaning electric nasal aspirator. The gear is the suction gear of the aspirator pump 12. The gear is switched to the second gear when short pressed once, the gear is switched to the third gear when short pressed once again, the gear is switched to the first gear when short pressed once again, and the gear is switched back to the second gear when short pressed once again. The gear is switched back and forth as above.

Optionally, the housing 11 is further provided with a state indicator 111. The state indicator 111 is connected to the electronic control module 16 and configured to indicate at least one of the operating state and the state of charge of the self-cleaning electric nasal aspirator.

It is to be noted that the preceding are merely embodiments of the present disclosure and the principles used therein. It should be understood by those skilled in the art that the present disclosure is not limited to the embodiments described herein. Those skilled in the art can make various apparent changes, adaptations, and substitutions without departing from the scope of the present disclosure. Therefore, although the present disclosure has been described in detail through the preceding embodiments, the present disclosure is not limited to the preceding embodiments and may include more other equivalent embodiments without departing from the concept of the present disclosure. The scope of the present disclosure is determined by the scope of the appended claims.

What is claimed is:

1. A self-cleaning electric nasal aspirator, comprising:
a suction assembly comprising a suction nozzle and a storage compartment, wherein the suction nozzle is in communication with the storage compartment, and the storage compartment is configured to temporarily store nasal mucus sucked out through the suction nozzle,
wherein the suction assembly further comprises a suction compartment and a storage compartment cover, the storage compartment cover is disposed at an upper end of the storage compartment, a liquid storage space is formed by being enclosed by the storage compartment cover and the storage compartment, and the suction compartment is sleeved outside the storage compartment and the storage compartment cover.

2. The self-cleaning electric nasal aspirator according to claim 1, further comprising a principal machine, wherein a front end of the principal machine is detachably connected to the suction assembly, and the principal machine comprises a housing, an aspirator pump and a buffer compartment; and
the housing is a hollow structure, the aspirator pump is disposed in a cavity of the housing, the aspirator pump is a water and gas dual-purpose pump, the buffer compartment is disposed at a rear end of the housing, the buffer compartment is connected to a discharge port of the aspirator pump, and a bottom of the principal machine is provided with an outlet through which the buffer compartment is in communication with an outside of the principal machine; and
the storage compartment is in communication with a suction port of the aspirator pump, and the aspirator pump is configured to pump, into the buffer compartment, nasal mucus overflowing from the storage compartment.

3. The self-cleaning electric nasal aspirator according to claim 2, wherein the principal machine further comprises a connector, the aspirator pump is connected to the storage compartment through the connector, the connector is a hollow structure, the connector is provided with a first pipe and a second pipe in communication with the first pipe, the first pipe is connected with, in a pluggable manner, an eduction pipe at a bottom of the storage compartment, and the second pipe is connected with the suction port of the aspirator pump in a pluggable manner.

4. The self-cleaning electric nasal aspirator according to claim 3, wherein the suction compartment is provided with a suction pipe, one end of the suction pipe extends into the liquid storage space through the storage compartment cover, and the suction nozzle is sleeved at another end of the suction pipe.

5. The self-cleaning electric nasal aspirator according to claim 4, wherein a first gap is disposed between the suction pipe and the storage compartment cover, a second gap is disposed between an outer wall of the storage compartment and an inner wall of the suction compartment and between an outer wall of the storage compartment cover and the inner wall of the suction compartment, a liquid guiding passage is disposed at the bottom of the storage compartment, and the liquid storage space is in communication with the eduction pipe through the first gap, the second gap and the liquid guiding passage.

6. The self-cleaning electric nasal aspirator according to claim 4, wherein the suction compartment, the storage compartment and the storage compartment cover are each made of a transparent or translucent material.

7. The self-cleaning electric nasal aspirator according to claim 3, wherein the storage compartment is made of a transparent polycarbonate material, and the connector is made of a soft rubber material.

8. The self-cleaning electric nasal aspirator according to claim 2, wherein the principal machine further comprises a power supply module, the power supply module comprises a battery, the battery is configured to supply power to the aspirator pump, a back of the housing is provided with a battery compartment, and the battery is accommodated in the battery compartment.

9. The self-cleaning electric nasal aspirator according to claim 8, wherein the battery is a rechargeable battery, the principal machine further comprises a charging module, the charging module is configured to charge the battery, the charging module comprises a type-c socket, and the type-c socket is disposed on the housing.

10. The self-cleaning electric nasal aspirator according to claim 2, wherein the principal machine further comprises an electronic control module, the electronic control module comprises a control circuit board and a control button, the control circuit board is disposed in the cavity of the housing, the control circuit board is electrically connected to the aspirator pump, the control button is disposed on the control circuit board, and the control button penetrates through a front side of the housing.

11. The self-cleaning electric nasal aspirator according to claim 10, wherein the housing is further provided with a state indicator, and the state indicator is connected to the electronic control module and configured to indicate at least one of an operating state or a state of charge of the self-cleaning electric nasal aspirator.
